(19) United States
(12) Patent Application Publication
(10) Pub. No.: US 2024/0374171 A1
(43) Pub. Date: Nov. 14, 2024

(54) Method and System of Non-invasive Optical Rotation Angle Sensing Polarimeter for Aqueous Glucose Concentration Measurement

(71) Applicant: Allied Biotech Development Inc., Taoyuan (TW)

(72) Inventors: Chien Chou, Taipei (TW); Chi-Tse Chao, Taichung (TW); Lidek Chou, Tustin, CA (US)

(21) Appl. No.: 18/660,013

(22) Filed: May 9, 2024

Related U.S. Application Data
(60) Provisional application No. 63/577,626, filed on May 9, 2023.

(51) Int. Cl. A61B 5/145; A61B 5/1455

(52) U.S. Cl. CPC A61B 5/14532; A61B 5/14558; A61B 2562/0233

(57) ABSTRACT

Disclosed is a system of non-invasive optical rotation angle sensing polarimeter for aqueous glucose concentration measurement. A linear polarization angle is set at 45° along propagation axis, the polarized beam transmitting aqueous humor and the polarized beam splitter whose polarization directions are orthogonal at 0° and 90° of rotation in the directions of propagation axes; difference of intensities of the orthogonal polarization components is measured in a balanced detector configuration able to obtain Stokes vector component S₁ which relates to the optical rotation angle and aqueous glucose concentration under common noise rejection mode, the sum of the intensities to normalize S₁ enables reduction of the optical intensity fluctuation noise too.

### CROSS REFERENCE TO RELATED APPLICATIONS

The application claims priority to and the benefit of U.S. provisional patent application Ser. No. 63/577,626, filed May 9, 2023, which is hereby incorporated by reference in its entirety.

### FIELD

The present disclosure relates to a method and a system of non-invasive optical rotation angle sensing polarimeter for aqueous glucose concentration measurement, and a method and a system of non-invasive optical rotation angle sensing polarimeter for measuring optical activity of an optically active medium

### BACKGROUND

With increase of average life expectancy, the number of people suffering from age-related diseases, such as diabetes, also grows. Particularly for those with diabetes, they would suffer from complications affecting the functioning of multiple organs in the course of disease progression, resulting in significant burdens in terms of medical resources and health care. The most important thing for people with diabetes to do to prevent further worsening of the conditions is strict monitoring and management of blood glucose levels; therefore, a patient's blood glucose concentration information needs to be obtained regularly on a daily basis. In this regard, an important technical problem to be solved is how to accurately and non-invasively measure blood glucose levels.

Aqueous humor of the eye is mainly formed from the blood filtrated through the blood-aqueous barrier. The aqueous glucose concentration is highly correlated with the glucose concentration in the blood, with a time delay. In addition, due to optical activity of glucose, a beam, after passing through the glucose, has its linear polarization angle rotated and changed due to optical activity, where the rotation extent is directly proportional to the glucose concentration. As such, it is reasonably expected to obtain the aqueous glucose concentration by measuring an amount of rotation of the linear polarization angle of the emergent light emerging from the aqueous humor on which the linearly polarized light is incident. However, the cornea-linear birefringence induced elliptical polarization effect on the linearly polarized light should also be taken into account when measuring the aqueous glucose concentration; due to cornea-linear birefringence effect, when a typical linearly polarized light passes through the cornea, an elliptical polarization is produced, causing degradation of detection sensitivity, so that the aqueous glucose concentration cannot be effective measured.

Furthermore, every chemical substance has a unique absorption spectrum; this is why existing non-invasive glucose measurement approaches do not favor measuring aqueous glucose concentration; instead, they deploy a light source and an optical receiver on the exterior of human skin to measure glucose level non-invasively by measuring the scattering of the incident light beam and the absorption spectrum of skin tissues to determine a glucose concentration in a human body. However, the accuracy and sensitivity of the existing methods are limited due to uncertainty caused by varying with the subject's physiological conditions, such as skin surface temperature and incident angle of light beam. Meanwhile, intensity change of the light source over time and the environment change during measurement would also produce an inaccurate result, so that those relevant methods can only serve as a reference for personal health care purposes rather than for the purposes of diagnosis in clinics.

A solution for aqueous glucose concentration measurement has been described in U.S. Pat. No. 7,627,357 B2, as illustrated in FIG. 1. An optically coherent laser beam 90 is incident to the eye, obtaining emergent light 92 including the scattering, reflection, and transmission beams. By use of a Rayleigh filter 94, the Raman scattering spectral component in the emergent light is measurable with a Raman spectrometer 96, while separately processing, by use of an optical polarimetric system 98, for glucose concentration using the emergent light. Different Raman spectra or amounts of optical rotation of the linear polarization angle are generated in the emergent light when the laser beam is irradiated onto the glucose molecules and small protein molecules such as albumins in the aqueous humor, while the measurement signal of the Raman spectrum is so weak, only of a one-millionth order of the incident light, so that the Raman spectral signal actually cannot be measured via aqueous humor; if the intensity of the incident light is increased to a measurable extent, the eye would be damaged seriously, which is practically impossible to implement.

Another solution has been disclosed in US 2017/0020385 A1, as illustrated in FIG. 2, in which light emitted by a multi-wavelength (λ1, λ2, λ3 . . . ) light source 80 is incident into the eye via a polarizer 82, the emergent light passes through a polarimetric analyzer 84, detectors 86, 87 measure the various wavelengths λ1, λ2, λ3 . . . one by one to obtain a first group of measurement data with respect to the various wavelengths, and finally an electronic signal processor 88 solves a set of optimum values through extensive processing using the non-linear least squares method, whereby aqueous glucose concentration readings without cornea-contributed birefringence effect are obtained.

However, the above method requires multiple wavelengths of the beam in the separate measurement; the multiple wavelengths of the incident and emergent light beams are required during independent measurements which are time-consuming so that a real-time measurement becomes impossible. This causes inconvenience to human test. Even worse in the course of wavelength shifting, the environment and operating conditions change over time, particularly the intensity and phase of light source also change over time. These significantly affect the reliability of the measurement and resulting in uncertainties and errors in the measurement of aqueous glucose concentrations.

In view of previous discussion, a non-invasive glucose monitoring system for diabetic managing conditions in real time becomes very important. Obtaining accurate aqueous glucose concentration in real time particularly under a consideration of the linear birefringence of cornea becomes the most interesting research topic for diabetes. In addition, the method based on optical rotation angle detection of a linear polarized light may also be applied to the protein structure analysis which is in terms of the optical rotation dispersion (ORD) using multiple wavelengths of the light. Therefore, the optical activity and the spectrum analysis of optically active media play a very important role such as in the precise chemical reaction detection and the present disclosure may provide a real-time and highly sensitively method on optical activity measurement.

### SUMMARY

In view of the above, an objective of the present disclosure is to provide a system of non-invasive optical rotation angle sensing polarimeter for aqueous glucose concentration measurement. It is based on an optical polarimetry under an optical balanced detector configuration for an optical rotation angle detection of a linear polarization beam in real time. This present disclosure enables reduction of the common background noises including the thermal induced electronic noise and environmental disturbance background noise simultaneously. As results, the detection sensitivity may be improved significantly.

Another objective of the present disclosure is to provide a system of non-invasive optical rotation angle sensing polarimeter for aqueous glucose concentration measurement, which, by splitting an emergent light into polarized beam components whose polarization directions are orthogonal to each other and alternatively processing, effectively cancels out common background noise, thereby improving measurement sensitivity.

Another objective of the present disclosure is to provide a system of non-invasive optical rotation angle sensing polarimeter for aqueous glucose concentration measurement which, by adopting a single-wavelength polarized incident light, achieves efficient measurement of aqueous glucose concentration.

Another objective of the present disclosure is to provide a method of non-invasive optical rotation angle sensing polarimeter for aqueous glucose concentration measurement, which, by virtue of an ingenious optical arrangement, significantly simplifies processing of measured data, thereby effectively improving processing speed while reducing environment-induced time-varying interference.

Another objective of the present disclosure is to provide a method of non-invasive optical rotation angle sensing polarimeter for aqueous glucose concentration measurement, which, by splitting an emergent light into two polarized beam components whose polarization directions are orthogonal to each other and alternatively processing, effectively cancels out common background noise, thereby improving measurement accuracy and sensitivity of aqueous glucose concentration in real time.

Another objective of the present disclosure is to provide a system of non-invasive optical rotation angle sensing polarimeter for measuring a linearly polarized optical rotation angle and optical activity of an optically active medium, the system being configured to measure a linearly polarized optical rotation angle and optical activity of an optically active medium in a transparent sample substance, which, by virtue of different light source modulation methods and optical structure setup, obtains accurate concentration readings.

Another objective of the present disclosure is to provide a method of non-invasive optical rotation angle sensing polarimeter for optical activity measurement, which allows for measurement with a single-wavelength light source to rapidly measure optically activity, or obtains spectral properties of an optically active medium via multi-wavelength measurement.

To achieve the above and other objectives, there is provided a system of non-invasive optical rotation angle sensing polarimeter for aqueous glucose concentration measurement, the system being configured to measure aqueous glucose concentration, cornea at front surface of an eye having a fast axis and a slow axis due to the optical properties of cornea with linear birefringence, wherein the system comprises: a linear-polarization collimated light source configured to emit an incident polarized beam at a linear polarization start angle traveling along a light wave propagation axis (Z-axis), the incident polarized beam transmitting through the cornea into aqueous humor and emerging from the cornea to produce an emergent polarized beam whose polarization angle is rotated with respect to the incident polarized beam, wherein the linear polarization start angle of the incident polarized beam is defined at 45-degree rotation angle in X-Y plane which is perpendicular to the Z-axis; at least one first polarized beam splitter configured to split the emergent polarized beam into two routes of emergent polarized beam components whose polarization directions are orthogonal to each other, wherein the two routes of emergent polarized beam components have their respective light wave propagation axes (Z-axis and Z′-axis), the light wave propagation axes thereof being angled to each other, and the two routes of emergent polarized beam components are polarized by a 0-degree rotation angle along X-axis in X-Y plane and by a 90-degree rotation angle along Y′-axis in X′-Y′ plane respectively; at least two photodetectors configured to measure optical intensities of the two emergent polarized beam components, respectively, wherein the emergent polarized beam components measured by the two photodetectors have their respective polarization directions orthogonal to each other; and an electronic signal processor configured to measure the difference of the intensities of the two routes of emergent polarized beam components to obtain Stokes vector component S₁ as a function of an optical rotation angle of the emergent polarized light beam, whereby the common background noises are reduced effectively. In the meantime, the setup is configured to measure the sum of the optical intensities of the two emergent polarized beam components whose polarization directions are orthogonal to each other as a norm value to normalize the Stokes vector component S₁, whereby the slow time-varying intensity fluctuation noise of the incident polarized beam can be reduced efficiently.

There is also provided a method of non-invasive optical rotation angle sensing polarimeter for aqueous glucose concentration measurement, the method being configured to measure aqueous glucose concentration where the cornea presenting the linear birefringence properties having a fast axis and a slow axis results in an elliptical polarization of the incident polarized beam. This method comprises: a) emitting, by a linear-polarization collimated light source, an incident polarized beam which transmits through the cornea of the eye into aqueous humor and emerges from the cornea to produce an emergent polarized beam whose polarization angle is rotated with respect to the incident polarized beam; b) measuring intensity of the emergent polarized beam via a feedback control loop to control the polarization angle of the linear-polarization collimated light source until the intensity of the emerging polarized beam is maximized. As result, the linear polarization angle of the linear-polarization collimated light wave is aligned with the fast axis (or slow axis) of the cornea; c) rotating the X-Y coordinates of the first polarized beam splitter that the X-axis and Y-axis are in the direction at 45-degree angle from the linear polarization start angle of the incident collimated beam and then splitting the polarized beam into two emergent polarized beams whose polarization directions are orthogonal to each other; d) measuring a difference optical intensities of the two emergent polarized beam components by use of photodetectors and a signal processor for the Stokes vector component S₁ to thereby reduce the common background noise at high efficiency. The Stokes vector component S₁ is a function of the optical rotation angle produced by aqueous humor; and e) measuring a sum of the optical intensities of the two emergent polarized beam components whose polarization directions are orthogonal to each other simultaneously as a norm value in order to normalize the Stokes vector component S₁ thereby to remove slow time-varying intensity fluctuation noise of the incident laser beam.

With the methods and systems noted supra, the present disclosure can obtain Stokes vector components S₀, S₁ and S₂ simultaneously by use of a single-wavelength, collimated linearly polarized light beam of which the linear polarization at angle θ₀ is properly adjusted. S₁ is obtained by measuring the difference of optical intensities of two components whose polarization directions are along 0° (X-axis) and 90° (Y-axis) directions of the linear polarization. They are orthogonal to each other. So does the S₂ by measuring the difference of light intensity of two components whose polarization directions are along +45° and −45° directions from X-axis respectively. They are orthogonal to each other too. Meanwhile, Sₒ is the norm value by measuring the sum of optical intensities of two orthogonal polarization components simultaneously and S₁ and S₂ are normalized by Sₒ separately. In the setup, the emergent light resulting from a polarized beam splitter to divide into two routes of components whose polarization directions are orthogonal to each other; in this way, the magnitude of rotation angle of the incident polarization light beam highly correlates with the aqueous glucose concentration and can be obtained from S₁ or from S₂ directly where the incident linear polarized light beam of the polarization angle θ₀ is properly adjusted. In addition, since the present disclosure can obtain the Stokes vector components S₀, S₁ and S₂ simultaneously in a simple and precise manner as illustrated in FIG. 3, the cornea-induced linear birefringence parameters can be further calculated by means of the normalized S₁ and S₂ simultaneously. Thus, the cornea induced elliptical polarization of the emergent polarized light beam can be compensated rapidly by way of a linear birefringence compensator via a feedback signal, Finally, allowing for the emergent polarized components becoming a rotated linear polarization and exhibits the angle of rotation of the linearly polarized beam in FIG. 7. This enables converting the rotation angle into the aqueous glucose concentration directly and precisely.

Therefore, the measurement and calculation process as disclosed herein does not require using a complicated optical setup, without considerable noises and measuring errors. Moreover, it does not consume much processing time either during measurement; due to the overall simple structure and rapid measurement capability, the environment time-varying background noises are significantly reduced based on the balanced detector configuration for measuring the Stokes vector components, so that the systems and methods disclosed herein can be a highly sensitive optical rotation angle sensing polarimeter in terms of the normalized S₁ and S₂ not only for accurate measurement of the optical activity of an optical active medium but also for non-invasive aqueous glucose concentration detection for diabetes. The present disclosure may also employ light sources modulated by modes such as intensity modulation, amplitude modulation, and phase modulation; and by introducing modulators with corresponding modulation modes in the setup during measurement, the optical activity of an optically active medium can be measured simply and rapidly, and the measurement results not only agree with predicted values but also are accurate as shown in FIG. 6; moreover, due to the capability of improving sensitivity, a better result was performed by compensating the elliptical polarization during measurement shown in FIG. 9, thereby being capable of applying this method in Analytical Chemistry or Biosensors industries for biomedical applications.

### BRIEF DESCRIPTION OF THE DRAWINGS

FIG. 1 is a schematic diagram of an existing optical structure and operation thereof,

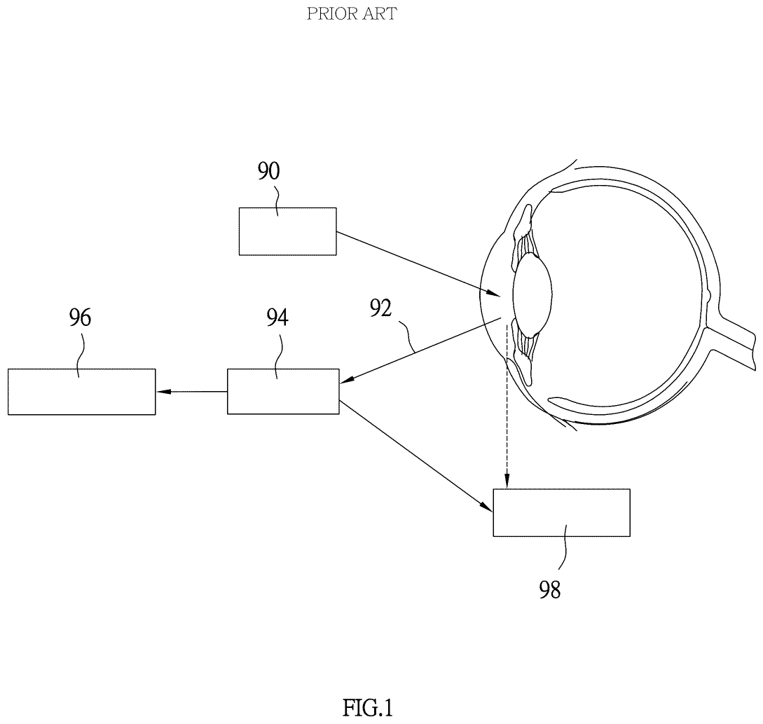

FIG. 2 is a schematic diagram of another existing optical structure and operation thereof;

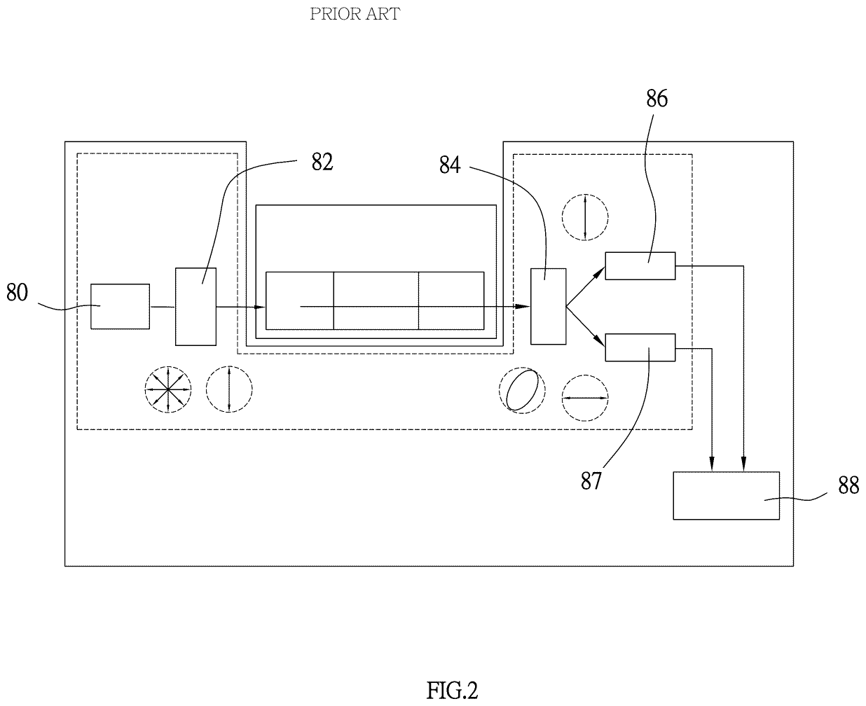

FIG. 3 is a schematic optical structure diagram of the first implementation of a system of non-invasive optical rotation angle sensing polarimeter for aqueous glucose concentration measurement according to the present disclosure;

FIG. 4 is a flow diagram of a method corresponding to the implementation illustrated in FIG. 3, illustrating operation of the optical rotation angle sensing polarimetric system of the implementation;

FIG. 5 is a stereoscopic diagram of an emerging beam in the implementation illustrated in FIG. 3, illustrating that the emergent polarized beam is split by the first polarized beam splitter into two routes of emergent polarized beam components whose propagation directions are mutually perpendicular and whose polarization directions are mutually orthogonal;

FIG. 6 is a two-dimensional distribution diagram of the measured data resulting from application of the implementation of FIG. 3, illustrating relationships between measured optical intensity data and the glucose concentrations in tri-distill water solution;

FIG. 7 is a schematic optical structure diagram of the second implementation of a system of non-invasive optical rotation angle sensing polarimeter for aqueous glucose concentration measurement, illustrating an optical structure integrated with a linear birefringence compensator;

FIG. 8 is a flow diagram of a method corresponding to the implementation illustrated in FIG. 7, illustrating operation of the optical rotation angle sensing polarimetric system of this implementation;

FIG. 9 is a two-dimensional distribution diagram of the measured data resulting from application of the implementation of FIG. 7, illustrating relationships between measured optical intensity data and the glucose concentrations in tri-distill water solution;

FIG. 10 is a schematic optical structure diagram of the third implementation of a system of non-invasive optical rotation angle sensing polarimeter for aqueous glucose concentration measurement according to the present disclosure, illustrating an optical structure of a reflection type polarimetric system;

FIG. 11 is a schematic optical structure diagram of the fourth implementation of a system of non-invasive optical rotation angle sensing polarimeter for aqueous glucose concentration measurement according to the present disclosure, illustrating an optical structure of a transmission polarimetric system integrated with a quarter wave plate and mutually orthogonal circular polarizers;

FIG. 12 is a schematic optical structural diagram of an implementation of a polarimetric system of measuring optical activity of an optically active medium;

FIG. 13 is a top view of the emergent beam in the implementation of FIG. 12, illustrating that the emergent polarized beam is split by a polarized beam splitter into two routes of emergent polarized beam components whose propagation directions are not perpendicular but polarization directions are orthogonal.

### DETAILED DESCRIPTION OF EMBODIMENTS

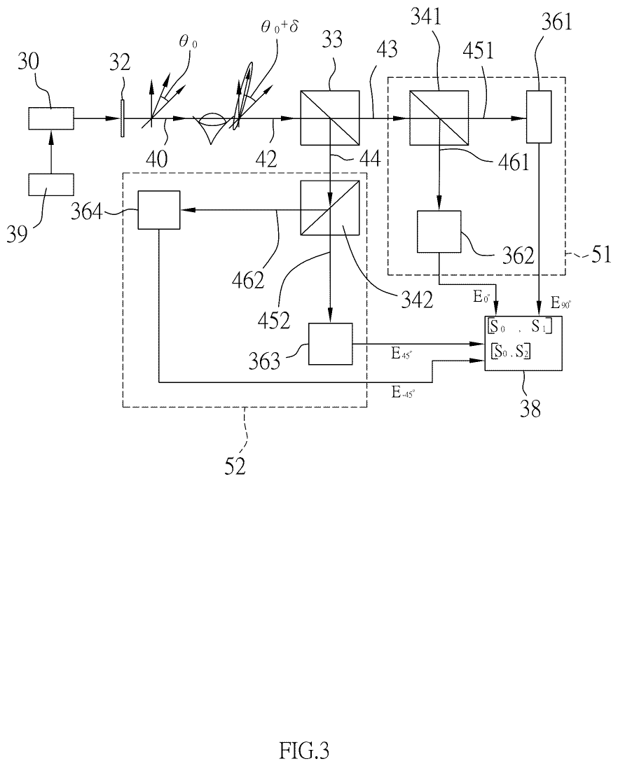

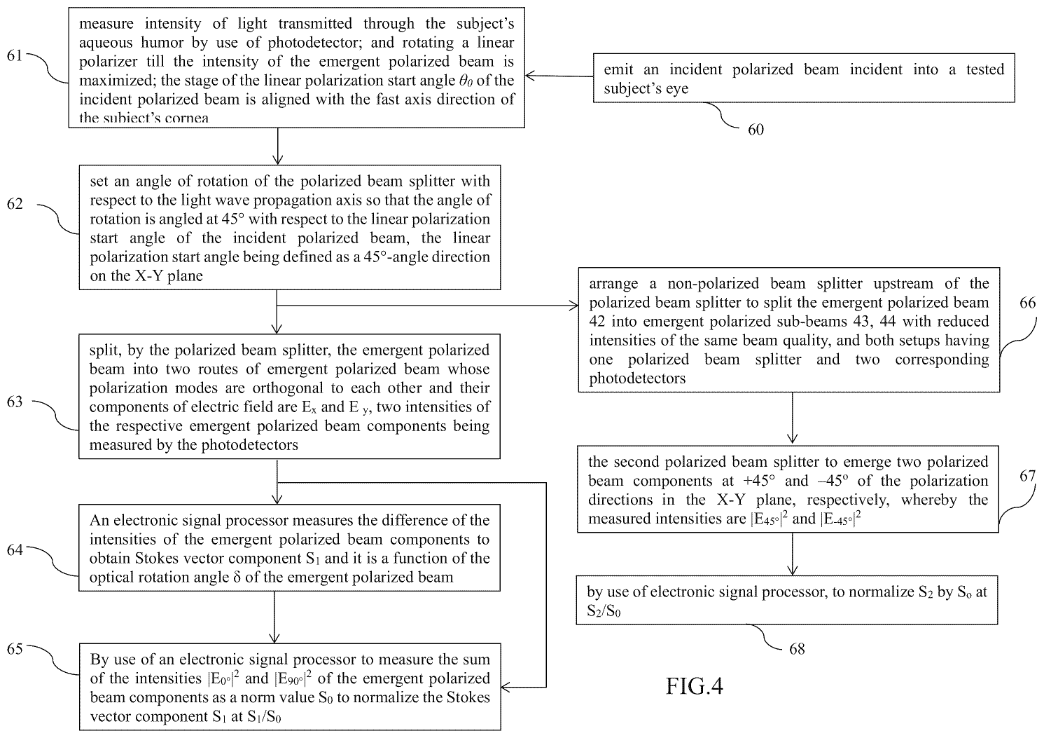

FIGS. 3 and 4 illustrate the first implementation of the present disclosure, in which a collimated light source 30 is exemplarily a laser source; the collimated light source 30, modulated by use of an optical modulator 39, emits an intensity modulated laser beam 40; after passing through a linear polarizer 32, an incident linear polarized beam 40 is incident into and transmits through the eye of test subject (step 60); then, the incident polarized beam 40 transmits through the cornea and aqueous humor to form an emergent polarized beam 42; since the cornea presenting linear birefringence properties that results in different propagation speeds of the linear polarization light beam along the fast axis and slow axis, and then the emergent polarized light beam 42 becomes elliptically polarized. At the same time, the aqueous humor introduces optical rotation of the emergent polarized light beam 42 a small angle with respect to the incident polarized beam 40 of which the polarization angle at θ₀ is properly setup. After the emergent polarized beam 42 is directed into a polarized beam splitter (50:50) 341, it divides, based on the electric field of linear polarization along X-axis and Y-axis, into emergent polarized beam components Eₓ and E_{y} of 451, 461 respectively. Their polarization directions are orthogonal on optical intensity; and then they are measured by using photodetectors 361, 362, respectively.

Defining that the plane which is perpendicular to the propagation direction of light beam is the X-Y plane and the X-axis is set along a given direction, theoretically, the state of polarization of a polarized light wave can be characterized precisely by the Stokes vector given below:

$$( \begin{matrix}{S}_{0} \\ {S}_{1} \\ {S}_{2} \\ {S}_{3}\end{matrix} ) = ( \begin{matrix}{\vert {E}_{x} \vert}^{2} + {\vert {E}_{y} \vert}^{2} \\ {\vert {E}_{x} \vert}^{2} - {\vert {E}_{y} \vert}^{2} \\ 〈 {E}_{x} {E}_{y}^{*} 〉 + 〈 {E}_{x}^{*} {E}_{y} 〉 \\ - j ( 〈 {E}_{x} {E}_{y}^{*} 〉 - 〈 {E}_{x}^{*} {E}_{y} 〉 )\end{matrix} ) = ( \begin{matrix}{\vert {E}_{x} \vert}^{2} + {\vert {E}_{y} \vert}^{2} \\ {\vert {E}_{x} \vert}^{2} - {\vert {E}_{y} \vert}^{2} \\ {\vert {E}_{45 °} \vert}^{2} - {\vert {E}_{- 45 °} \vert}^{2} \\ {\vert {E}_{r} \vert}^{2} - {\vert {E}_{l} \vert}^{2}\end{matrix} )$$

Step 61 in FIG. 4 is the first step of calibration of the present disclosure in which a collimated single wavelength light beam is transmitted through aqueous humor of eye and the output intensity is measured by use of photodetectors 361, 362, respectively, and the linear polarizer 32 is rotated continuously until that the output intensity of the emergent polarized beam 42 becomes maximized; by then, the linear polarization start angle 60 of the incident polarized beam 40 is aligned with the fast axis (or slow axis) direction of the cornea as disclosed in US 2005/0154269 A1 and WO 2005/067522 A2. This calibration procedure can be accomplished by use of a feedback control module. In this implementation, alignment with the slow axis can be setup too. However, in the present disclosure, the linear polarization angle of the incident polarized beam 40 is defined at θ₀ equal to 45° from X-axis based on the X-Y coordinate setup in calculation procedure. Even if the propagation direction of the beam is directed to different direction, the X-Y coordinates for sensing optical rotation angle of the linear polarized beam following the definition of Z-axis is along the light beam propagation direction and the X-Y coordinates is on the plane perpendicular to Z-axis.

In step 62, the first polarized beam splitter 341 is rotated an angle at 45° along the light wave propagation axis such that the directions of X-axis and Y-axis of the polarized beam splitter 341 are 45° from the incident linear polarization start angle at 60. And then the X-Y plane of polarized beam splitter 341 is defined precisely. Without linear birefringence effect from cornea, and at zero concentration of the glucose solution, the intensities of the emergent linear polarization components of 451, 461 of the electric field Eₓ and E_{y} respectively from a polarized beam splitter (50:50) 341 would be equal to the intensities measured by the photodetectors 361, 362 accordingly.

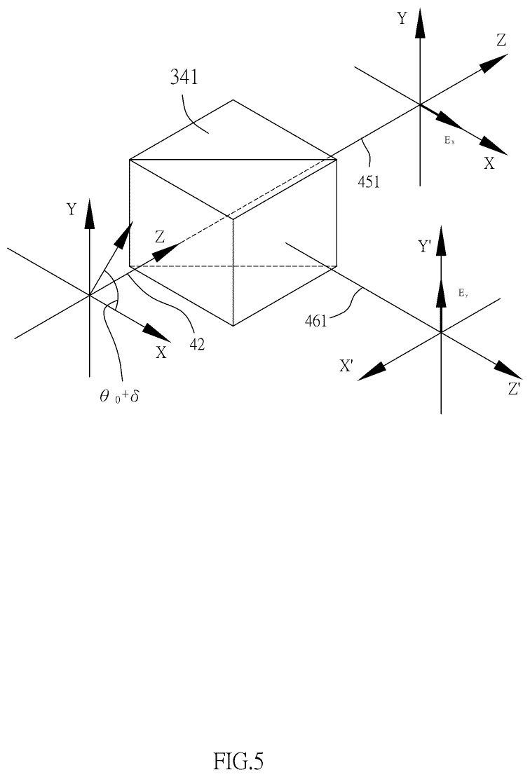

The first polarized beam splitter 341 in this implementation is exemplarily a cubic 50:50 linear polarized beam splitter illustrated in FIG. 5. In step 63, the emergent polarized beam 42 is split by the first polarized beam splitter 341 into two emergent polarized beam components Eₓ and E_{y} 451, 461 whose propagation directions are perpendicular to each other and whose polarization modes are orthogonal to each other. In this implementation, the beam propagation direction of the emergent polarized beam component 451 is consistent with the initial emergent polarized beam 42, while the beam propagation direction of the emergent polarized beam component 461 is perpendicular to the beam propagation direction of the initial emergent polarized beam 42. When the polarization directions of respective emerging polarizing beam components 451, 461 are taken into account, their corresponding X-Y and X′-Y′ planes are characterized based on the Z-axis and Z′-axis, respectively. They are physically consistent to each other. As a result, the first polarized beam splitter 341 divides into the emergent polarized beam component 451 which is responsible for Eₓ of the linear polarization in X-axis, while the emergent polarized beam component 461 is only for E_{y} of the polarization in the Y′-axis. As results, the intensities of the emergent polarized beam components Eₓ and E_{y} 451, 461 measured by corresponding photodetectors 361, 362 are |Eₓ|² and |E_{y}|² respectively. This result can be extended into the setup of using different types of polarized beam splitters such as the Wollaston prism of which the propagation directions of two emergent polarized beam components which are responsible for Eₓ and E_{y} respectively, are not perpendicular to each other.

Next, in step 64, an electronic signal processor 38 is used to measure a difference of the intensities of the emergent polarized beam components 451, 461 to obtain Stokes vector component S₁ as a function of the optical rotation angle δ of the linear polarized beam. Since the measurement setup employed herein is a balanced detector configuration that performs the common background noise reduction mode including the electronic noise and the environmental disturbance noise enabling to be effectively reduced during measurement. In addition, a large dynamical range of the measurement on optical rotation angle at high detection sensitivity and at high accuracy is anticipated as well. In addition, the scattering and depolarization effects by the tested medium can be reduced in this setup too. In step 65, the electronic signal processor 38 can also obtain the sum of |Eₓ|² and |E_{y}|² of the emergent polarized beam components simultaneously in order to obtain the Stokes vector component Sₒ as a norm value to normalize the Stokes vector component S₁, so that the slow time-varying optical intensity fluctuation noise from light source can be removed effectively at S₁/S₀.

A collimated single wavelength light source 30 integrated with a linear polarizer 32 emits a linear polarization light beam 40 of the linear polarization starting angle θ₀ at 45° is generated where the X-Y coordinates is defined by both X-axis and Y-axis are +45° and −45° from the linear polarization start angle of the linear polarization light beam 40 respectively. The incident linear polarized light beam 40 is incident into glucose solution, so that an optical rotation angle δ is generated in the polarization angle of the emergent polarized beam where

$${E}_{x} = A cos ( {θ}_{0} + δ )$$

$$and$$

$${E}_{y} = A sin ( {θ}_{0} + δ )$$

A denotes the amplitude of the incident linearly polarized beam 40. The optical polarimeter in the present disclosure can measure S₁ given by

$${S}_{1} = {\vert {E}_{x} \vert}^{2} - {\vert {E}_{y} \vert}^{2} = {A}^{2} [ {cos}^{2} ( {θ}_{0} + δ ) - {sin}^{2} ( {θ}_{0} + δ ) ] = {A}^{2} cos 2 ( {θ}_{0} + δ )$$

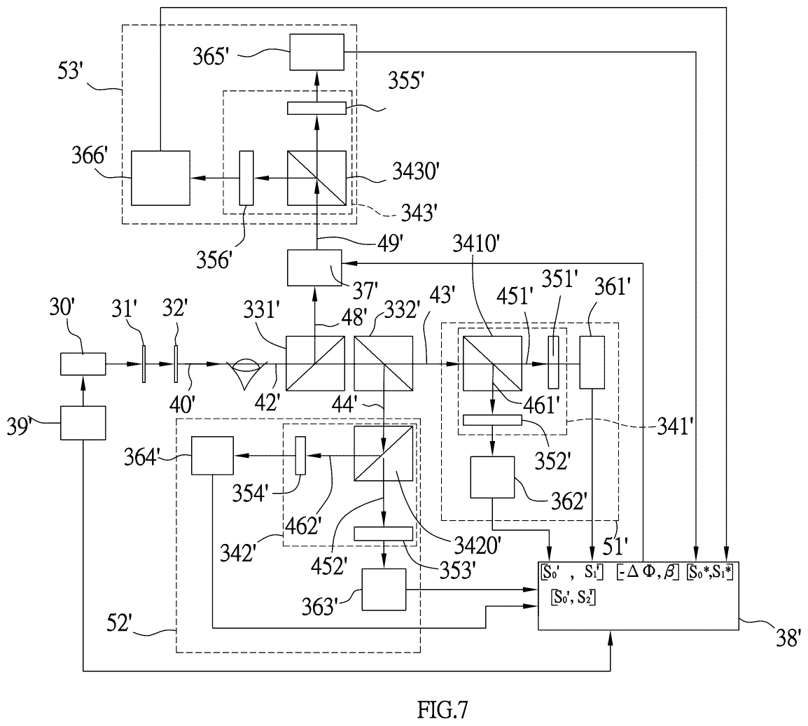

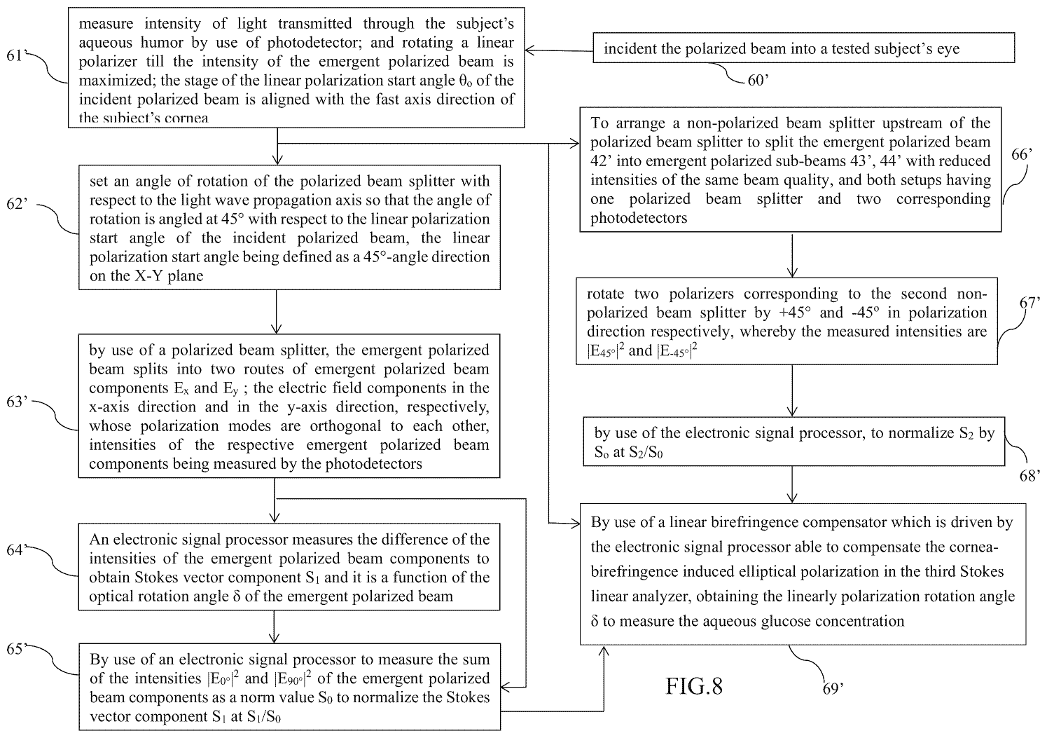

Since the linear polarization start angle θ₀ is set at 45° in direction (or −45° in direction) on the X-Y plane that 2θ₀ equals to ±90°, so that S₁=A²|sin 2δ|≈2A²|δ|, which is proportional to the optical rotation angle and directly relates the glucose concentration in solution. Furthermore, S₁ can be normalized by the norm value of S₀=|Eₓ|²+|E_{y}|²=A²[cos²(θ₀+δ)+sin²(θ₀+δ)]=A²; thus, the optical rotation angle δ is measurable by S₁/Sₒ directly at high detection sensitivity. This method can be further applied to aqueous glucose concentration detection for diabetes once the linear birefringence effect of the cornea is compensated properly by setting up the polarimetric system in FIG. 7 and following the measurement steps in FIG. 8 accordingly

Furthermore, in procedure subsequent to step 62, step 66 of arranging a non-polarized beam splitter 33 upstream of the first polarized beam splitter 341 may be added. In step 66, the non-polarized beam splitter 33 is used to split the emergent polarized beam 42 into emergent polarized sub-beams 43, 44 of same optical properties, and a second polarized beam splitter 342 of a similar optical setup is arranged to split the emergent polarized sub-beam 44 into emergent polarized beams 452, 462, which are measured by photodetectors 363, 364 corresponding to the second polarized beam splitter 342, respectively. For simplicity, such an optical setup is referred to as a Stokes vector analyzer 52; the Stokes vector analyzer in the present disclosure has been significantly simplified compared with the existing Stokes vector analyzers available in the market. In other words, the emergent polarized beam 42, after passing through the non-polarized beam splitter 33, is split into emergent polarized sub-beams 43, 44, which are then directed to the Stokes vector analyzers 51, 52, respectively, as illustrated in FIG. 3. The Stokes vector analyzer 52 differs from the Stokes vector analyzer 51 only in step 67 in which the second polarized beam splitter 342 of two orthogonal polarization directions are 45° and −45° in polarization directions; as such, the intensities measured by the photodetectors 363, 364 will be |E_{45°}|² and |E_{-45°}|², respectively. Apparently,

$${S}_{2} = 〈 {E}_{x} {E}_{y}^{*} 〉 + 〈 {E}_{x}^{*} {E}_{y} 〉 = {\vert {E}_{45 °} \vert}^{2} - {\vert {E}_{- 45 °} \vert}^{2} = 2 {A}^{2} [ cos ( {θ}_{0} + δ ) sin ( {θ}_{0} + δ ) ] = {A}^{2} sin 2 ( {θ}_{0} + δ )$$

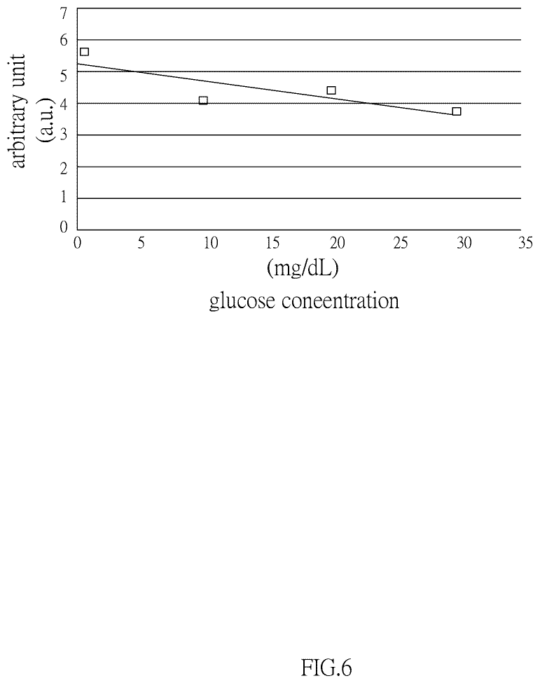

Also, in step 68 similar to step 65, the electronic signal processor 38 may obtain S₂ and the normalized S₂/S₀. S₀=|E_{45°|}²+|E_{-45°}|²=A². The system of non-invasive optical rotation angle sensing polarimeter according to the first implementation of the disclosure can obtain very stable readings, as illustrated in FIG. 6 where the actual uncertainty of measurement is smaller than the spot size of the measurement as such the error bars cannot be revealed in FIG. 6; therefore, the accuracy of the measurement in this implementation satisfies the requirements under the operating environment. When 2θ₀ happens to be 0° or 180°, S₂=A²|sin 2δ|≈2A²|δ|. Thus the pair of S₀ and S₂ is measured simultaneously, enabling measurement of the optical rotational angle of the linear polarization light beam at high detection sensitivity following same approach as described previously for S₁ on aqueous glucose concentration measurement.

FIGS. 7 and 8 illustrate a system of non-invasive optical rotation angle sensing polarimeter for aqueous glucose concentration measurement. According to a second implementation of the disclosure, in which all elements identical to the preceding implementation, such as the color filter 31′, the polarizer 32′, the non-polarized beam splitter 332′, and the subsequent first and second polarized beam splitters 341′, 342′, the photodetectors 361′, 362′, 363′, 364′, the emergent polarized beams 42′, 43′, 44′, the emergent polarized beam components 451′, 452′, 461′, 462′, and the Stokes vector analyzers 51′, 52′ are all represented with similar reference signs to the preceding implementation, with the same operating flow employed, i.e., comprising steps 60′ . . . 68′. The main difference lies in that in this implementation, the collimated light source 30′ is a light emitting diode (LED), and in step 60′, the LED, which is modulated by modulator 39′, emits an incident polarized beam 40′ with intensities varying periodically, so that the intensities as measured exhibit a synchronous periodical variation. In addition, the first and second polarized beam splitters 341′, 342′ in this implementation are set up by a non-polarized beam splitter 3410′ integrated with linear polarizers 351′, 352′, and a non-polarized beam splitter 3420′ integrated with linear polarizers 353′, 354′, respectively.

The Stokes vector analyzers 51′, 52′ in FIG. 7 may simultaneously measure S₀, S₁ and S₂ at the same time. Theoretically, the output electric field of the emergent polarized light beam which transmits to the aqueous humor and cornea of eye of tested subject can be calculated by using following equations where ΔΦ is the phase retardance and β is the azimuth angle of the fast axis of the cornea due to linear birefringence. According to the step of the measurement of aqueous glucose concentration in FIG. 8 which is based on the setup of present disclosure in FIG. 7, where the incident linear polarization light beam of the polarization angle is aligned parallel to the fast axis of the cornea and the linear polarization angle θ₀ is set at 45°, the electric field of the emergent polarized light beam is expressed by

$$( \begin{matrix}{E}_{x} \\ {E}_{y}\end{matrix} ) = [ {R}^{- 1} ( β ) ( LB ( Δ ϕ ) ) R ( β ) ] ( \begin{matrix}cos δ & sin δ \\ - sin δ & cos δ\end{matrix} ) ( \begin{matrix}1 \\ 1\end{matrix} )$$

$$R ( β ) = ( \begin{matrix}cos β & sin β \\ - sin β & cos β\end{matrix} )$$

$$LB ( Δ ϕ ) = ( \begin{matrix}{e}^{i \frac{Δ ϕ}{2}} & 0 \\ 0 & {e}^{- i \frac{Δ ϕ}{2}}\end{matrix} )$$

Thus, the normalized S₁ and S₂ are measured simultaneously in FIG. 7. They are expressed as the following:

$$[ {S}_{1} ] / [ {S}_{0} ] = {sin}^{2} ( \frac{Δ ∅}{2} ) sin 4 β ( δ ∼ 0 )$$

$$[ {S}_{2} ] / [ {S}_{0} ] = 1 - {sin}^{2} ( \frac{Δ ∅}{2} ) ( 1 + cos 4 β ) ( δ ∼ 0 )$$

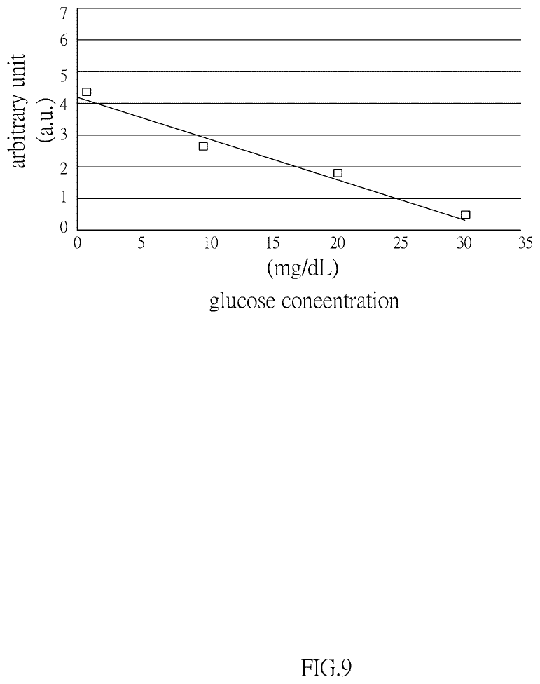

As results, the linear birefringent parameters ΔΦ and β of the cornea can be calculated by above equations analytically. In this implementation, a non-polarized beam splitter 331′ disposed upstream of the non-polarized beam splitter 332′ and a third Stokes vector analyzer 53′ corresponding to the non-polarized beam splitter 331′ are further arranged; therefore, in step 69′ of the method according to this implementation, the electronic signal processor 38′ further outputs an elliptical parameter (−ΔΦ, β), driving a birefringence compensator 37′ to compensate for cornea linear birefringence induced elliptical polarization of the emergent polarized beam, so that the cornea linear birefringence induced elliptical polarization may be eliminated from the emergent polarized beam 48′ received in the third Stokes vector analyzer 53′, to result in an emergent polarized beam 49′ of the optical rotation angle δ of the linear polarization angle only by aqueous glucose in aqueous humor. The emergent polarized beam 49′ likewise passes through the polarized beam splitter 343′ constructed by the non-polarized beam splitter 3430′ integrated with the linear polarizers 355′, 356′ and is measured by the photodetectors 365′, 366′, respectively, leading to desired measurement results. The test results are illustrated in FIG. 9. Compared with FIG. 6 where without birefringence compensator being setup, the glucose concentrations measured by the optical setup of FIG. 7 not only shows same resolution but also performs higher accuracy of the glucose concentration in a tri-distill water solution. This result implies that the implementation of FIG. 7 satisfies the requirement for the non-invasive aqueous glucose concentration measurement. Extensively, the Stokes vector analyzers 51′, 52′ in FIG. 7 may also be modified by using two mutually orthogonal circular polarizers to measure [S₃]/[S₀]=sin ΔØ cos 2β (δ-0) in real time, and then corneal birefringent parameters may be obtained based on the real-time measured S₀, S₁, S₂, and S₃.

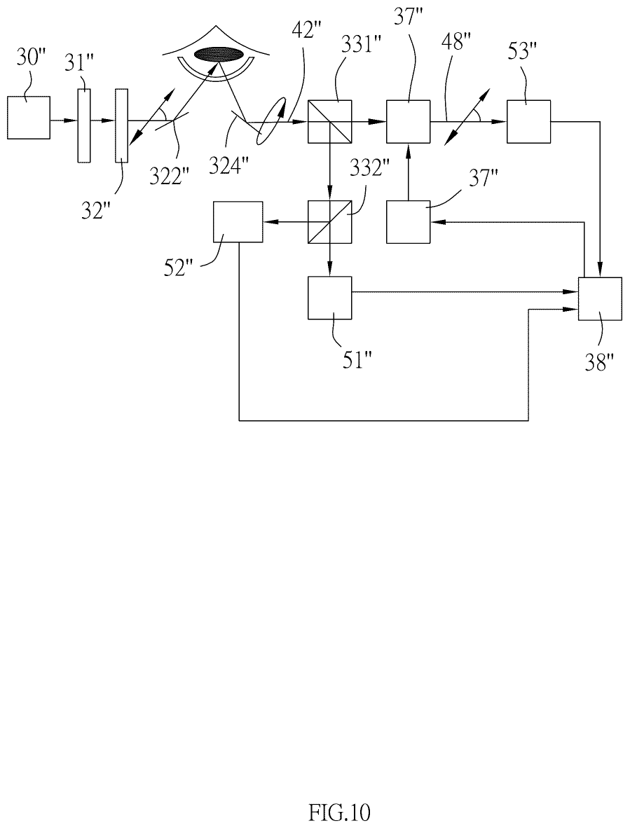

Although the linear-polarization collimated light sources in the preceding implementations are all based on the light beam transmitting through the aqueous humor of eye, those skilled in the art would easily appreciate that the optical configuration may also change to receive the polarized light beam emerging reflectively from the aqueous humor of the eye. For example, in a third implementation of the disclosure illustrated in FIG. 10, an illuminating light source 30″ of a mobile phone is adopted, where a color filter collimator 31″ and a linear polarizer 32″ are additionally mounted in front of the light source 30″ to thereby construct a linear-polarization collimated light source; a pair of optical reflector 322″, 324″ are provided, for which the light is incident on eye and is reflected back as the reflected emergent polarized light beam and is measured by the non-polarized beam splitters 331″, 332″ and the Stokes vector analyzers 51″, 52″ which are identical to the preceding implementations, whereby Stokes vector components S₀, S₁, S₂ are obtained; likewise, the electronic signal processor 38″ calculates the cornea birefringence parameters (ΔΦ, β) of the emergent polarized light beam, which are then compensated by the linear birefringence compensator 37″, so that the emergent polarized beam 48″ acquired by the third set of Stokes vector analyzer 53″ has induced elliptical polarization removed, whereby a simplified, portable system of non-invasive optical rotation angle sensing polarimeter for aqueous glucose concentration measurement is set up.

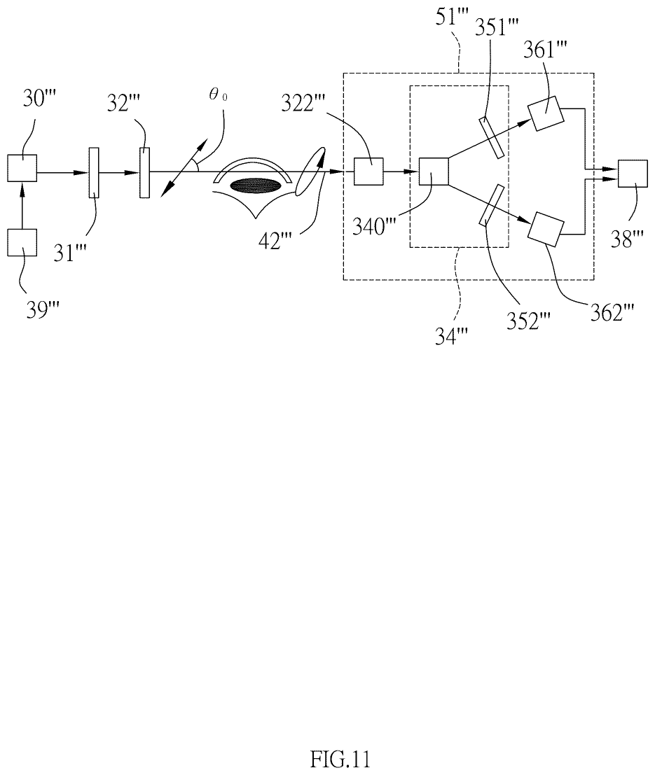

Furthermore, although all of the preceding implementations measure the intensities of two mutually orthogonal linear polarization components of the emergent polarized beam, a person of normal skill in optics would easily appreciate that in a fourth implementation of the disclosure, the emergent polarized beam may also be converted and split into a left-hand emergent circularly polarized beam component and a right-hand emergent circularly polarized beam component by use of a quarter wave plate. The polarization directions of two circular polarized beam components are orthogonal to each other; as illustrated in FIG. 11, the beam, which is emitted by the collimated light source 30″′ modulated by the modulator 39″′, passes through the color filter 31″′ and the linear polarizer 32″′ to produce a linearly polarized incident polarized beam 40″′ which is incident upon the tested subject's eye; a quarter-wave plate 322″′ is added in the pathway of the transmitted emergent polarized beam 42″′; then, the emergent polarized beam after 322″′ passes through the non-polarizing beam splitter 340″′ and a circularly polarized beam splitter 34″′ constructed by a left-hand circular polarizer 351″′ and a right-hand circular polarizer 352″′ which are orthogonal to each other and are divided into a left-hand circularly polarized component Eₗ and the right-hand circularly polarized component Eᵣ, with the intensities |Eₗ|² and |Eᵣ|² of the emergent polarized beam components being measured by photodetectors 361″′, 362″′, respectively; as given in the equation below, the Stokes vector components S₀, S₁ may be likewise derived by the electronic signal processor 38″′ through addition/subtraction processing, whereby the aqueous glucose-contributed optical rotation δ of the polarization angle is further expressed as:

$$( \begin{matrix}{S}_{0} \\ {S}_{1} \\ {S}_{2} \\ {S}_{3}\end{matrix} ) = ( \begin{matrix}{\vert {E}_{r} \vert}^{2} + {\vert {E}_{l} \vert}^{2} \\ {\vert {E}_{r} \vert}^{2} - {\vert {E}_{l} \vert}^{2} \\ 〈 {E}_{r} {E}_{l}^{*} 〉 + 〈 {E}_{r}^{*} {E}_{l} 〉 \\ - j ( 〈 {E}_{r} {E}_{l}^{*} 〉 - 〈 {E}_{r}^{*} {E}_{l} 〉 )\end{matrix} )$$

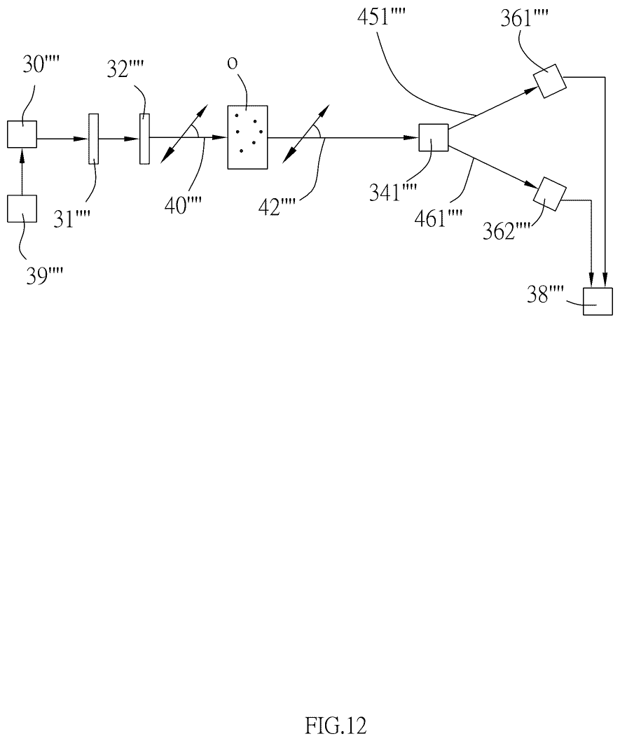

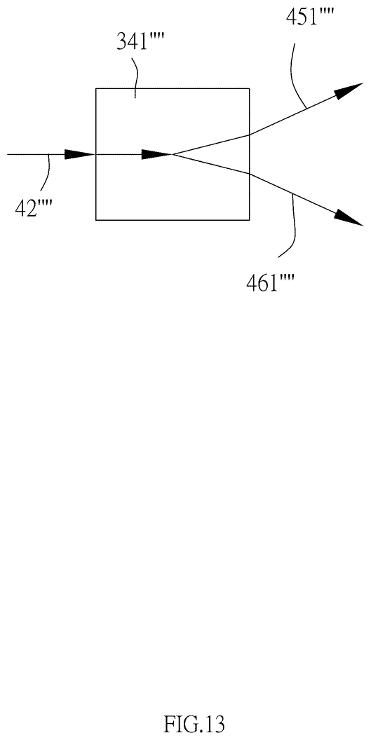

Of course, without influence from the birefringent material such as cornea, the method of non-invasive optical rotation angle sensing polarimeter may also be applied to protein structure analysis based on optical rotation dispersion (ORD) in spectroscopy or the dynamical measurement of optical activity of an optically active medium in real time. The optical activity analysis according to a fifth implementation of the disclosure is illustrated in FIG. 12 and FIG. 13, in which a collimated light source 30″″ modulated by a modulator 39″″ emits a light, the light passing through the color filter 31″″ and the linear polarizer 32″″ to form an incident linearly polarized beam 40″″ that is directed into a sample substance O, whereby an emergent polarized beam 42″″ is formed; likewise, a linear polarization angle of the incident linearly polarized beam 40″″ is defined as a linear polarization start angle 60. After the emergent polarized beam 42″″ is irradiated into a polarized beam splitter 341″″, the emergent polarized beam 42″″ is divided into components Eₓ and E_{y} 451″″, 461″″ whose polarization directions are orthogonal to each other based on the components of linear polarization; afterwards, the intensities of the Eₓ and E_{y} components 451″″, 461″″ are measured and outputted by corresponding photodetectors 361″″, 362″″, respectively; finally, the intensities are likewise processed by the electronic signal processor 38″″. In addition, the first polarized beam splitter 341″″ in this implementation is illustrated in FIG. 13, which is a Wollaston prism; although the two routes of emergent polarized beam components 451″″, 461″″ split thereby have their polarization directions orthogonal to each other, the propagation directions of their light wave propagation axes are not perpendicular to each other. Furthermore, the spectroscopy of optical activity of a specific sample substance may also be constructed by real-time measurement of the rotation angles with multiple wavelengths and this can then be applied to precisely characterize specific constitutions in the sample substance.

What have been described above are only implementations of the disclosure, which are not intended for limiting the implementation scope of the disclosure; any equivalent changes and modifications made to the claims of the present application and the contents of the specification shall fall within the scope of the disclosure. After having read the description of the implementations of the disclosure, those skilled in the art will appreciate that the disclosure is novel, inventive and practical and represents notable progress.

I/We claim:

1. A system of non-invasive optical rotation angle sensing polarimeter for aqueous glucose concentration measurement, the system being configured to measure aqueous glucose concentration, cornea at front part of an eye having a fast axis and a slow axis due to properties of linear birefringence, wherein the system comprises:
a linear-polarization collimated light source configured to emit an incident polarized light beam at a linear polarization angle and propagation along a light wave propagation axis, the incident polarized light beam transmitting through the cornea into aqueous humor and emerging from the cornea to produce an emergent polarized light beam whose polarization angle is rotated with respect to the incident polarized beam, wherein the linear polarization start angle of the incident polarized beam is defined as a 45-degree rotation angle in a propagation direction along the light wave propagation axis;
at least one first polarized beam splitter configured to split the emergent polarized beam into two routes of emergent polarized beam components whose polarization directions are orthogonal to each other, wherein the two routes of emergent polarized beam components have their respective light wave propagation axes, the light wave propagation axes thereof being angled to each other, and the two routes of emergent polarized beam components are polarized by a 0-degree rotation angle and a 90-degree rotation angle along their respective light wave propagation axes, respectively;
at least two photodetectors configured to measure intensities of the two routes of emergent polarized beam components, respectively, wherein the emergent polarized beam components measured by the two photodetectors have their respective polarization directions orthogonal to each other;
and an electronic signal processor configured to measure the difference of the intensities of the two routes of emergent polarized beam components to obtain Stokes vector component S₁ as a function of an optical rotation angle with respect to the incident linear polarization start angle which is produced by an optical active medium, whereby common background noise is canceled out for enhancing detection sensitivity, and configured to measure a sum of the intensities of the two routes of emergent polarized beam components whose polarization directions are orthogonal to each other as a norm value to normalize the Stokes vector component S₁, whereby time-varying optical intensity fluctuation noise of the incident polarized light beam is reduced.

2. The system of claim 1, wherein the linear polarization start angle of the incident polarized beam is set to maximize intensity of the emergent polarized beam, allowing for the linear polarization start angle to be aligned with the fast axis of the cornea.

3. The system of claim 1, further comprising at least one non-polarized beam splitter which is disposed upstream of the first polarized beam splitter and configured to split the emergent polarized beam into a plurality of emergent polarized sub-beams, wherein the system further comprises a second polarized beam splitter which is the linear-polarization collimated light source, whereby one of the plurality of emergent polarized sub-beams is split into two routes of emergent polarized beam components oriented such that their polarization directions are orthogonal to each other and they are respectively at a 45-degree angle and a negative-45-degree angle in polarization directions, wherein the electronic signal processor measures the difference of the intensities of the two routes of emergent polarized beam components oriented respectively at the 45-degree angle and the negative-45-degree angle in polarization direction to obtain Stokes vector component S₂ as a function of an amount of rotation of the polarization angle, thereby canceling out common background noise, measures a sum of the intensities of the two routes of emergent polarized beam components whose polarization directions are orthogonal to each other as a normal value to normalize the Stokes vector component S₂, thereby canceling out the time-varying optical intensity fluctuation noise of the incident polarized beam, and calculates a cornea-induced birefringence parameters to output a compensation signal which is proportional to the birefringence parameter, the compensation signal including a phase retardance and a fast-axis azimuth angle.

4. The system of claim 3, further comprising a linear birefringence compensator configured to receive the compensation signal to compensate for a linear birefringence effect in the emergent polarized beam.

5. The system of claim 1, wherein the first polarized beam splitter, or the second polarized beam splitter, or each of the first polarized beam splitter and the second polarized beam splitter comprises a quarter-wave plate, a beam splitter, a left-hand circular polarizer, and a right-hand circular polarizer, so as to split the emergent polarized beam into a left-hand circularly polarized component and a right-hand circularly polarized component.

6. A method of non-invasive optical rotation angle sensing polarimeter for aqueous glucose concentration measurement, the method being configured to measure aqueous glucose concentration, cornea at front surface of an eye having a fast axis and a slow axis due to linear birefringence, wherein the method comprises:
emitting, by a linear-polarization collimated light source, an incident polarized beam which transmits through the cornea of the eye into aqueous humor and emerges from the cornea to produce an emergent polarized beam whose polarization angle has an amount of optical rotation with respect to the incident linear polarization start angle of the incident polarized beam;
arranging at least one first polarized beam splitter to split the emergent polarized beam into two routes of emergent polarized beam components whose polarization modes are orthogonal to each other;
measuring intensities of the two routes of emergent polarized beam components whose polarization directions are orthogonal to each other, measuring, by an electronic signal processor, a difference of the intensities of the two routes of emergent polarized beam components to thereby cancel out common background noise, obtaining Stokes vector component S₁ as a function of the amount of optical rotation of the polarization angle of the incident polarized beam; and
measuring a sum of the intensities of the two routes of emergent polarized beam components whose polarization directions are orthogonal to each other as a normal value S₀, and normalizing the Stokes vector component S₁ to thereby cancel out time-varying optical intensity fluctuation noise of the incident polarized beam.

7. The method of claim 6, further comprising: before the arranging at least one first polarized beam splitter, measuring, by a feedback control loop, intensity of the emergent polarized beam, and feeding back to control the linear-polarization collimated light source till intensity of the emergent polarized beam is maximum, allowing for the incident linear polarization start angle to be aligned with the fast axis of the cornea.

8. The method of claim 6, further comprising: before the arranging at least one first polarized beam splitter, splitting, by a non-polarized beam splitter disposed upstream of the first polarized beam splitter, the emergent polarized beam into a plurality of emergent polarized sub-beams;
splitting, by a second polarized beam splitter, one of the plurality of emergent polarized sub-beams into two routes of emergent polarized beam components oriented such that their polarization directions are orthogonal to each other and they are respectively at a 45-degree angle and a negative-45-degree angle in polarization directions; measuring, by the electronic signal processor, a difference of the intensities of the two routes of emergent polarized beam components oriented respectively at the 45-degree angle and the negative-45-degree angle in polarization directions to obtain Stokes vector component S₂ as a function of an amount of the optical rotation angle of the linear polarization angle, thereby canceling out common background noise, and measuring a sum of the intensities of the two routes of emergent polarized beam components whose polarization directions are orthogonal to each other as a normal value to normalize the Stokes vector component S₂, thereby canceling out time-varying optical intensity fluctuation noise of the incident polarized beam;
calculating, by the electronic signal processor, a cornea-induced birefringence effect based on Stokes vector components S₀, S₁, and S₂ to output a compensation signal proportional to the cornea-induced birefringence effect.

9. A system of non-invasive optical rotation angle sensing polarimeter for optical activity measurement of an optical active medium, the system being configured to measure optical activity of the optical active medium in a transparent sample substance, wherein the system comprises:
a periodically modulated, linear-polarization collimated light source configured to emit an incident polarized beam at a linear polarization start angle propagating along a light wave propagation axis, the incident polarized beam transmitting through the transparent sample substance and emerging from the sample substance to produce an emergent polarized beam whose polarization angle is rotated with respect to the incident polarized beam, wherein the linear polarization start angle is defined as a 45-degree rotation angle in a propagation direction along the light wave propagation axis;
at least one polarized beam splitter configured to split the emergent polarized beam into two routes of emergent polarized beam components whose polarization directions are orthogonal to each other, the two routes of emergent polarized beam components whose polarization directions are orthogonal, the emergent polarized beam components being polarized at a 0-degree rotation angle and a 90-degree rotation angle in propagation directions along their separate light wave propagation axes, respectively;
at least two photodetectors configured to measure intensities of the two routes of emergent polarized beam components, respectively;
and an electronic signal processor configured to measure a difference of the intensities of the two routes of emergent polarized beam components to obtain Stokes vector component S₁ as a function of an optical rotation angle with respect to the incident linear polarization start angle which is produced by the optical active medium, whereby the common background noise is cancelled out for enhancing detection sensitivity, and configured to measure a sum of the intensities of the two routes of emergent polarized beam components whose polarization directions are orthogonal to each other as a norm value to normalize the Stokes vector component S₁, whereby time-varying optical intensity fluctuation noise of the incident polarized beam is reduced.
